(19) United States
(12) Patent Application Publication
(10) Pub. No.: US 2003/0003133 A1
(43) Pub. Date: Jan. 2, 2003

(54) Oral delivery of allergens in milk

(76) Inventor: Heinz Schneider, Cordast (CH)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/182,298

(22) PCT Filed: Jan. 31, 2001
(86) PCT No.: PCT/EP01/01029

(30) Foreign Application Priority Data
Feb. 2, 2000 (GB) 0002386.1

(51) Int. Cl. A61K 39/00; A61K 39/36; A61K 47/00

(52) U.S. Cl. 424/439; 424/184.1; 424/275.1

(57) ABSTRACT

The present invention relates to the use of milk and milk-related products as a carrier or delivery vehicle for allergens in the process of oral tolerance induction. In one preferred embodiment, infants are subjected to primary oral tolerance through provision of a cocktail of allergens in infant milk formula. In another preferred embodiment, secondary oral tolerances is induced through administration of one or more allergens mixed with milk to a mammal of any age. The benefits of using milk as a carrier are evident from experiments which show that ovalbumin tolerance can effectively be induced in an allergic dog model at low dosages. A possible explanation for this is that milk stabilizes the allergen and presents it to the immune system in such a way that it is very potent in effecting a tolerance response.

### FIELD OF THE INVENTION

The present invention relates to the use of milk as a carrier for allergens to induce oral tolerance to these allergens, to compositions comprising milk, as a carrier, together with one or more allergens, and to the use of such compositions to treat and/or prevent allergic conditions.

### BACKGROUND OF THE INVENTION

Oral tolerance (OT) is a physiological process by which the body's immune responses to environmental allergens are controlled. It has been demonstrated that this natural mechanism can be harnessed to treat or prevent allergic syndromes by feeding appropriate antigens to induce non-responsiveness or reduced responsiveness of the immune system.

There are two categories of immune disorders in which induction of OT may be beneficial, namely allergic conditions and autoimmune diseases. The first category includes those diseases in which patients manifest inappropriate, deteriorated immune responses (allergen specific IgE antibodies, the mediators of allergic reactions) to ubiquitous antigens, and which may result from the failure of natural OT induction, at least in the case of food allergies. OT induction may be applied as a prophylactic or therapeutic treatment of such diseases. The second category consists of autoimmune disorders, which do not arise from the failure of natural OT induction, but from breakdown of systemic self tolerance. Patients who have developed auto-immune disorders are potential candidates for therapeutic treatment by OT induction.

In theory, OT induction can be utilized as a means of preventing the development of allergic conditions in high risk individuals (primary OT), or as a therapeutic treatment for established syndromes (secondary OT). In practice, OT induction is currently not in routine use for treatment or prevention of allergies, and modern therapeutic approaches rely on anti-inflammatory/immunosuppressive therapy or the treatment of symptoms with steroids or anti-histamines. However, immunosuppressive agents do not provide a satisfactory treatment for allergies since they are non-specific and are not targeted to any particular tissue or antigen. As a result they are responsible for a multitude of undesirable side effects. Further, both anti-inflammatory and immunosuppressive drugs can provide only short-term relief from allergic symptoms. In the future OT induction will undoubtedly supersede immunosuppressive therapy in the treatment of allergies, since it is long-lasting, specific, safe, affordable, and has few or no side-effects.

OT induction has been exploited in a number of animal and human studies, to show that intentional oral dosing of an allergen can result in hyporesponsiveness to a later challenge with the same allergen. Rodents, especially mice, are a popular choice of experimental organisms for investigating allergies, but in fact mice do not suffer naturally from allergic symptoms. Therefore the predictability of results in mouse models for human allergic diseases is questionable. In contrast, dogs, like humans, suffer from allergic diseases (e.g. atopic dermatitis) and it is therefore expected that results from dog studies are predictable for man. Thus, recently-established colonies of atopic, genetically high responder IgE allergic dogs are proving to be an invaluable tool in research into allergy (de Weck et al. Prakt Tierarzt 1998;79:6-23; and de Weck et al. Allergologie 2, 1999;2:92-97). It has been shown that primary IgE tolerance can be induced in these dogs in the first 6 months of life.

One eventual aim is to apply the promising results obtained in such studies to develop effective preventative treatments for allergies in susceptible individuals. Targets for prevention include the common allergic syndromes, such as hay-fever, asthma, allergic rhinitis, eczema, nut allergies, anaphylactic shock, nickel hypersensitivity, atopic dermatitis (including contact dermatitis), gastrointestinal allergies etc, and also auto-immune diseases, in which the body fails to distinguish between self and non-self, including multiple sclerosis, systemic lupus erythematosus (SLE), uveitis, rheumatoid arthritis, type I diabetes, Grave's disease and Crohn's disease. However, before OT induction can be applied to the general public the techniques must be further refined in order to ensure that they are absolutely safe and consistently successful.

Previous attempts at inducing oral tolerance have either not employed a carrier, or have relied on the use of a limited selection of delivery systems, such as water, saline, gelatin capsules, orange juice, or the multiple emulsion systems described by Elson et al in Oral Tolerance, Mechanisms and Applications, Annals of the New York Academy of Sciences 1996, Volume 778, pp 156 to 162. However, not all antigens can easily be dissolved in these carriers, and further, many antigens delivered in carriers are digested within the body and thereby rendered ineffective.

What has been conspicuously lacking in the field until now has been the availability of a cheap, readily available, natural, safe, and extremely effective delivery vehicle for antigens for use in the induction of oral tolerance. We have surprisingly found that all of these requirements are met through the use of milk from any mammalian species, or milk substitutes, as an antigen carrier. When provided in milk, antigens appear to be presented to the immune system in such a way that they are extremely effective in inducing a tolerance reaction.

### SUMMARY OF THE INVENTION

According to a first aspect of the present invention there is provided use of milk as a carrier for one or more exogenous allergens for inducing oral tolerance.

According to a second aspect of the invention a composition is provided which comprises a mixture of milk with one or more exogenous allergens.

According to a third aspect, the invention provides a process for the preparation of a composition comprising milk and one or more exogenous allergens, comprising the step of mixing said milk with said one or more allergen(s).

According to a further aspect of the invention there is provided use of a composition comprising milk and one or more exogenous allergens in the manufacture of a medicament or nutritional formulation for the induction of oral tolerance to said one or more allergen(s).

According to yet another aspect of the invention there is provided a method of treatment or prevention of an allergic or auto-immune condition by oral tolerance induction, which comprises administering to an individual in need of such treatment an effective amount of a composition comprising milk as a carrier and one or more exogenous allergens responsible for said allergy or auto-immune condition.

### BRIEF DESCRIPTION OF THE DRAWINGS

FIG. 1 shows a comparison over 21 weeks of the ovalbumin IgE titers (measured by ELISA) in control subjects and in those dogs which have been treated by OT induction with ovalbumin.

FIG. 2 shows a comparison over 21 weeks of the ovalbumin IgG titers (measured by ELISA) in control subjects and in those dogs which have been treated by OT induction with ovalbumin.

### DETAILED DESCRIPTION OF THE INVENTION

As used herein, the term “oral tolerance” has the meaning which is widely understood among immunologists, namely a state of hyporesponsiveness to a particular antigen or antigens induced through contact of the antigen with the extensive mucosal surfaces, such as by ingestion of the antigen(s). In the present context the terms “antigen” and “allergen” are used interchangeably. Oral tolerance may be induced to just one antigen at a time, or simultaneously to a mixture of different antigens. For the purposes of describing the present invention “endogenous” allergens in the milk carrier are defined as those proteins or peptides naturally present in the milk which have a tendency to elicit allergic responses. “Exogenous” allergens, on the other hand, are synthesized or otherwise prepared separately from the milk carrier, and are mixed in with the milk carrier for intentional exposure to the immune system.

The quantity of an individual antigen which must be ingested in order to cause oral tolerance to that antigen may vary between individuals and according to the antigen in question (i.e. its antigenicity), but the skilled man can readily determine the ranges of antigen quantities required to elicit the desired response in any particular case. Suitable amounts for OT induction may be in the range 0.01 to 20 mg antigen/ml milk, preferably 0.05 to 4.5 mg/ml, more preferably 0.1 to 3 mg/ml, and most preferably 0.1 to 1 mg/ml milk. Suitable volumes of the antigen/milk mixture constituting a single dosage may be 5 to 500 ml, preferably 10 to 250 ml, and most preferably 20 to 100 ml. The total daily dosage of antigen is preferred to be less than 1000 mg, preferably in the range 1 mg to 500 mg, and most preferably 10 mg to 100 mg. In terms of amount of antigen in a single dosage per kg body weight the ranges are preferred to be 0.05 to 10 mg/kg, more preferably 0.10 to 5 mg/kg, and most preferably 0.15 to 3 mg/kg.

The term “milk” is used here in a broad sense, and is meant to describe any kind of mammalian milk or milk substitute. In the present context “milk substitute” means any kind of product ordinarily used in place of naturally-produced milk and which contains milk-derived proteins or peptides. This description encompasses processed milks and milk powders. Infant milk formula, expressed human milk, cow's milk and goat's milk are particularly well-suited carriers. Prior to mixing, the milk may be processed according to conventional methods, such as by pasteurization, homogenization, hydrolysis, dehydration, UHT methods and so on. Full cream (whole) milk, semi-skimmed milk and skimmed milk are all suited for use in accordance with the invention. Optionally, a hypoallergenic milk formulation, such as a milk formula based on hydrolyzed or partially-hydrolyzed milk proteins (e.g. Damira®, Adapta®) is used as the carrier.

According to a preferred embodiment of the invention an infant milk formula is used as a carrier for the induction of primary oral tolerance in infants below the age of one year. Infant milk formula is used as a substitute for breast milk and is provided in liquid form or as a powder for reconstitution with water. Local requirements and guidelines for infant formulae should be observed. These include European Commission Directives 91/321/EEC and 96/4/EC (amending 91/321/EEC), the guidelines published by the Ernährungskommission of the Schweizerische Gesellschaft für Pädiatrie, and the recommendations of the US FDA on the compositions of Term Infant Formulas. It is preferred that a cocktail of allergens is provided in infant milk formula so as to prevent a range of common allergic conditions, including hay fever, asthma, and the like.

A suitable formulation for use according to this embodiment of the invention comprises the following ingredients (and optionally others):

Energy content: 60 to 75 kcal/100 ml

Per 100 kcal:

Total fat content: 3.3 to 6.5 g

(linoleic acid content: 300 to 1200 mg)

Total protein content: 1.8 to 4.5 g

Total carbohydrate: 7 to 14 g

Calcium: minimum 60 mg

Phosphorus: 30 to 90 mg

Magnesium: 5 to 15 mg

Iron: 0.1 to 3 mg

Zinc: minimum 0.5 mg

Copper: 20 to 80 μg

Iodine: 5 to 75 μg

Sodium: 20 to 60 mg

Potassium: 60 to 200 mg

Chloride: 50-150 mg

Vitamin A: 60μg to 180 μg retinol equivalents

Vitamin D: 1 to 2.5 μg

Vitamin E: minimum 0.5 mg α-tocopherol

Vitamin K: minimum 4 μg

Vitamin B1 (thiamin): minimum 40 μg

Vitamin B2 (riboflavin): minimum 60 μg

Vitamin B3 (niacin): minimum 250 μg

Vitamin B6 (pyridoxine): minimum 35 μg

Vitamin B12 (cobalamin): minimum 0.1 μg

Folic acid: minimum 4 μg

Pantothenic acid: minimum 300 μg

Biotin: minimum 1.5 μg

Vitamin C (ascorbic acid): minimum 8 mg

The amount of allergen added to 100 kcal of this or a comparable milk formula is preferred to be in the range 1 to 500 mg, most preferably 10 to 150 mg.

For secondary oral tolerance induction the treatment usually involves oral administration of allergens to which the individual has already become sensitized, so it could be described as a therapeutic method rather than a prophylactic method. The aim in such circumstances would be to overcome an established allergic syndrome, generally in an older child (i.e. greater than 1 year old) or adult, by regular, repeat doses of the responsible allergen over a prolonged time-span. Therefore, according to a second embodiment of the invention, secondary tolerance is induced in a human or other animal by administering an exogenous allergen in a milk carrier, such as cow's milk. In the case of secondary oral tolerance induction the choice of antigen is made on the basis of allergies already manifested by the individual, so it is usual that a single allergen will be administered, or possibly two or three allergens together in the case of highly allergic, atopic, individuals. The preferred allergens for use in inducing secondary oral tolerance induction are allergens which would not normally be ingested, such as airborne allergens.

For inducing secondary oral tolerance the milk/antigen mixture of the invention may be processed into dairy products which would form part of a diet typical of an adult or older child, such as cheeses, yogurts, yogurt drinks, smoothies, milk-shakes, and the like. The benefits of the invention may be obtained provided that the milk and allergen are mixed together prior to any treatment which results in coagulation of the milk, since otherwise the allergen will not be sufficiently protected upon entry into the digestive system.

In the context of the present invention the milk is described as a carrier for allergens. In the present case “carrier” means an ingestible solid or liquid delivery vehicle in which allergens are dissolved or dispersed. When milk is used as a carrier, and depending on the type of milk used, oral tolerance may develop incidentally to antigens in the milk. Thus, in Example 1 oral tolerance to β-lactoglobulin is observed in treated dogs. Any oral tolerance arising incidentally in this way is additional to oral tolerance resulting from administration of the exogenous allergen(s). The exogenous allergen(s) is preferably not identical with any endogenous allergens present in the milk carrier. Alternatively, exogenous allergen(s) can be milk-derived antigen(s) which are identical with endogenous milk carrier allergens, but which are prepared separately and then mixed in with the milk carrier, such that the milk/allergen mixture is enriched in one or more specific milk allergens, and the mixture has a composition which is not identical with milk or milk substitutes as earlier defined. The milk carrier is ideally provided in significant weight or volume excess over the amount of exogenous allergen(s) which it carries, i.e. at least 100, preferably at least 1000 fold excess.

While it is not completely understood why antigens in milk should induce an oral tolerance response more effectively than they would do in tested agents such as water or gelatin capsules, the inventors' current opinions center on the role of coagulation of the milk around the antigen. This might afford the antigen complete or partial protection from digestion, and thereby preserve antigenic epitopes, or allow the creation of new epitopes which are recognized by the immune system in the intestinal tract as tolerogenic epitopes. Therefore a practical benefit of using milk as a carrier is that antigens which would otherwise be ineffective in inducing oral tolerance are able to do so when delivered in milk. Antigens which are not dependent per se on the presence of milk to be able to induce oral tolerance can nevertheless be effective in smaller dosages when milk is used in preference to other known carriers.

The compositions of the invention are obtainable by mixing exogenous allergen(s) with the milk carrier to create a solution, dispersion or suspension. In one suitable method of preparation of the compositions of the invention a small quantity of liquid (μl or ml quantities) or solid (mg quantities) antigen is added to a larger volume (usually 50 to 100 ml) of milk or dairy product, and if necessary shaking or stirring is performed in order to create a uniform mixture or solution. Optionally, the antigen(s) is mixed with, or dissolved in, the milk or dairy product immediately prior to feeding to a subject. However, it is preferred that milk and allergen are pre-mixed and stored as a mixture for sale and use as a combined composition. Pre-mixed antigen and milk can be supplied as a long-life product which does not require refrigeration, or as a chilled product with limited shelf-life, or as a frozen product. In some cases it may be desired to dehydrate or lyophilize a milk/antigen mixture for long-term storage and subsequent rehydration. Stable antigens should be able to survive such treatment and continue to be effective in inducing oral tolerance.

When a mixture of one or more allergens with infant milk formula or any other powdered milk formula is made on an industrial scale this is desirably achieved by blending the formula ingredients in the usual order, such as by pre-mixing carbohydrates and whey or casein proteins, and subsequently adding a blend of fat melted together with an emulsifier. If desired, trace elements, minerals and vitamins can then be added. The complete mixture is homogenized, pasteurized, and then spray-dried. Individual allergens which are relatively heat stable are preferably added to the blend after admixture of the fatty components of the formula. However, heat-labile allergens which are liable to denature or otherwise degrade under high pressure or at temperatures between about 30 and 50° C. are preferably dry-mixed with the spray-dried milk formula powder.

Certain allergies are particularly prevalent in modern Western society today, and it is these conditions which the present invention will generally be used to prevent and treat. Rarer conditions are also conveniently treated using the present therapeutic method. Treatable conditions include: hay-fever, asthma, allergic rhinitis, allergic conjunctivitis, eczema, nut allergies, anaphylactic shock, atopic dermatitis etc, and also auto-immune diseases, in which the body fails to distinguish between self and non-self, including multiple sclerosis, myasthenia gravis, diabetes mellitus, systemic lupus erythematosus (SLE), polychondritis, systemic scleroderma, Wegener's granulamatosis, dermatomyositis, chronic active hepatitis, psoriasis, Steven-Johnson syndrome, idiopathic sprue, uveitis (anterior and posterior), rheumatoid arthritis, arthritis chronica progediente, arthritis deformans, Grave's disease, autoimmune Inflammatory Bowel Disease (e.g. Crohn's disease and ulcerative colitis), sarcoidosis, primary biliary cirrhosis, keraconjunctivitis sicca, vernal keraconjunctivitis, uveitis of various forms, interstitial lung fibrosis, psoriatic arthritis and glomerulonephritis (with or without nephrotic syndrome, e.g. including idiopathic nephrotic syndrome or minimal change nephropathy).

Induction of oral tolerance to the antigens causing these allergies (either individually, or simultaneously when mixed as a cocktail together in the same portion of milk) is envisaged to become a preventative measure which children of a certain age would routinely undergo without pre-selection. In addition, on the basis of family history, location, likely future exposure to certain antigens and on various other factors, particular children can be singled out as being at high risk on the basis of their vulnerability to developing other allergies in later life, and these high risk children may benefit from oral exposure to antigens which only infrequently result in allergy in the general population. One such group of children are those having a genetic predisposition to developing a particular allergy or allergies in general. This group includes children having at least one allergic parent.

Oral induction may also be exploited for treating animals, such as household pets, and the present invention may be applied in the treatment or prevention of allergy in higher mammals, preferably companion animals such as dogs and cats.

It is preferred that repeat doses of the same milk/allergen mix will be orally administered over a time-period of days, weeks, or months. The allergen cocktail will normally be administered in daily dosages, but if preferred the interval between dosages may be extended or contracted to several hours or several days. A typical treatment period is 4 to 8 weeks. Small doses administered over relatively long time periods are generally preferred over larger doses administered over a short time-span. When small amounts of allergen are given to the patient on each occasion the risk of immunizing the patient against the allergen(s) is minimized.

Oral tolerance has been documented in a number of different mammalian species, and principally arises during early infancy, particularly during the first year of life, especially during weaning. The peak time for susceptibility to primary oral tolerance induction in humans is likely to be in the first 6 months after birth, preferably about 1 to 5 months after birth, most preferably 2 to 4 months after birth. Secondary oral tolerance induction is not linked to any particular age group. Due to variations between individuals, in some instances it may be desired to carry out an initial immune responsiveness test to determine whether the intended subject is likely to respond positively to antigen presentation as heretofore described. If so, the subject can be described as being susceptible to the induction of oral tolerance.

The allergen is preferably provided in substantially pure form for mixing with the milk. By “substantially pure” is meant generally at least 75% pure antigen contaminated with no more than 25% impurities, usually 75 to 90% pure antigen, preferably at least 90% pure antigen, more preferably at least 95% pure antigen, and most preferably at least 99% pure antigen. Purified proteinaceous antigens may be provided as intact protein, or converted into peptides through enzymatic digestion. It is also possible to provide the antigen in partially purified, enriched or concentrated form, for example as a bacterial protein extract from a recombinant source. Typical purification methods include protein isolation techniques known in the art, such as extraction, precipitation, chromatography, affinity chromatography, electrophoresis, and the like.

Alternatively, a relatively impure source of allergen can be used, such as for instance the supernatant from bacterial culture medium which contains secreted recombinant protein. Optionally, the source of allergen can be plants or material from plants which have been genetically engineered to express particular non-endogenous allergens. The advantage of allergen expression in plants is that large quantities of protein can be produced inexpensively without the need for special equipment, and the plant material, and protein extracted therefrom, is free of animal pathogens. In addition, plant-expressed antigens may be more soluble than the same antigens would be in their natural source material, where they may be sequestered. Furthermore, processing of proteins in plants can differ from that occurring in recombinant bacteria and yeast, which can have advantages in terms of recognition by the immune system. The allergens expressed in these plants may be targeted to the plant cell vacuole or to subcellular organelles, such as chloroplasts. The plant material may be used in the raw form or in the cooked form, but preferably raw. It is preferably processed so as to release the allergens into the milk to avoid the need for mastication during ingestion of the milk/antigen mixture. Preferably the expressed allergenic protein is extracted from the plant material (e.g. leaves) and then added to milk. Suitable plants include edible and non-edible crops, trees, algae, and particularly tobacco plants.

The invention does not encompass mixtures of milk with natural whole foodstuffs, or with natural foodstuffs which have not intentionally been processed or treated to extract their allergenic components for release into the milk. Furthermore, the allergens are preferably intact and/or native proteins or peptides, i.e. non-derivatised and unprocessed. For example, the allergens are preferably not products produced through processing by passage through the mammalian body.

Broadly, 4 classes of allergens may be included in the composition of the invention. These are: non-foodstuff, plant-originating antigens such as pollens; non-foodstuff, animal-originating antigens such as cat hair or dust-mite faeces; foodstuff allergens of any derivation; and environmental allergens not originating from plant or animal sources. In one embodiment of the invention none of the allergens carried in milk in accordance with the invention is a foodstuff allergen.

The following lists of antigenic materials, purified antigens and antigenic extracts includes examples of suitable antigens or sources of antigens which may be mixed with milk in accordance with the invention:

Foodstuffs: the milk allergens α₁-casein and β-lactoglobulin, milk whey protein, ovalbumin, egg, soy, cheese, fruits (e.g. strawberries, peaches), vegetables, yeast, maize, wheat, rice, legumes, peanuts, brazil nuts and other nuts, fish, shellfish, chicken, beef, pork, mutton, lamb etc.

Indoor airborne: house insect allergens, particularly house dust-mite and other arthropod faeces, flea allergens, epidermal elements (dander) and glandular elements from animals including human, mouse, rat, dog, cat, guinea pig, hamster, horse, pig, rabbit, sheep, cattle, birds, monkey etc.

Outdoor airborne: tree pollen (including alder, birch, oak, hazel, olive, hickory, elm, ash, pecan, box elder, cedar, plantain), mugwort, grass pollen (including timothy grass, Kentucky bluegrass, Johnson grass, Bermuda grass, redtop grass, orchard grass, sweet vernal grass and ryegrass pollen), weed pollens, especially ragweed, sagebrush, redroot pigweed, lamb's quarters, Russian thistle (tumbleweed) and English plantain, etc.

Moulds: Alternaria tennis, Aspergillus fumigatus, Cladosporium herbarum, Penicillium notatum, Candida albicans, Helminthosporium sp., Epicoccum sp., Fusarium sp., Mucor sp., Rhizopus sp., Aureobasidium (Pullaria) sp. etc.

Drug allergens: penicillin, aspirin, insulin etc.

Auto-antigens (for treatment of auto-immune disorders): collagen (type I, II or III), myelin basic protein or peptides, myelin proteolipid protein or peptides, interphotoreceptor binding protein, acetylcholine receptor, S-antigen, human or porcine insulin, S-antigen or peptides, human HLA peptides, glutamic acid dehydrogenase, glutamic acid decarboxylase, islet cell-specific antigen, acetylcholine receptor, MHC peptides, thyroglobulin etc.

Miscellaneous others: detergent enzymes, cosmetics, perfumes, latex, nickel, venom or stings from Hymenoptera (bees, wasps etc) and other insects, mosquito irritant proteins, reptile venom, viral allergens, proteinaceous contact allergens, poison ivy, poison oak, poison sumac, etc.

Of particular interest are antigens to which an individual would normally be exposed through inhalation in the form of an aerosol (“airborne” allergens or “aeroallergens”), such as grass and tree pollen antigens, fungal or mould spores, and house dust mite faeces. The preferred form of antigen is recombinant protein, usually purified away from the bacterial host and substantially free of endogenous bacterial protein.

Currently, various allergens which have been purified from recombinant sources are commercially available, including Bet v 1 (major birch pollen allergen); timothy pollen allergen, house dust mite allergens Der p I, Der p II, Der f I, and Der f II; Cte f1 (flea allergen) Can f II (a dog dander allergen); Lol p V (ryegrass allergen); Sor h I (major allergen from Sorghum halepense); ragweed pollen allergens Amb a I, Amb a I.1, and Amb a II; Aln g I(alder allergen); Cor a I(hazel allergen); Fel d I(a cat allergen), chains 1 and 2; bee venom PLA-2; peanut allergen Ara h 2 and mosquito allergens rAed a 1 and rAed a 2. Mature or truncated forms of these allergens may be used. It is preferred that cocktails of mixture of these allergens are prepared for mixing with milk, especially when inducing oral tolerance in infants.

The allergens of a cocktail can be pre-mixed prior to joint addition to the milk, or alternatively the allergens are added one by one to the milk. Various antigen cocktails are foreseen. By “cocktail” is meant any combination of two or more different antigens. A typical cocktail may contain 3 to 8 different allergens. Preferably each of the allergens in the cocktail is a different airborne allergen. For example, the cocktail may comprise:

(i) at least one outdoor airborne allergen; and

(ii) preferably also at least one indoor airborne allergen.

In this case the outdoor airborne allergen is preferably a grass or tree pollen allergen, while the indoor airborne allergen is selected from animal dander and house insect-derived allergens. A preferred outdoor airborne allergen is ragweed. Preferred indoor airborne allergens include house dust mite recombinant allergens Der f1 and Der f2 (mature and/or truncated), cat dander allergen Fel d1 (chains 1 and 2), and flea allergen Cte f1.

Alternatively, the cocktail may comprise:

(i) at least one animal dander allergen; and

(ii) at least one house insect allergen.

Ideally, the formulation of the invention consists essentially of milk and an amount of allergen which is sufficient to induce oral tolerance over repeated doses. However, it is foreseen that common food additives may also be included. Typical additives are those commonly added to milk formulations and dairy products, including emulsifiers, gelling agents, colorings, flavourings, lactic bacteria, sweeteners, preservatives, anti-oxidants, vitamins, minerals and trace elements.

If desired, an immune adjuvant may be added to the milk/allergen mixture. Suitable adjuvants for coadministration include lipopolysaccharides (LPS), and cholera toxin B chain.

The allergen(s) may be pre-incubated with thioredoxin before mixing with milk. Alternatively thioredoxin may be added to the milk or milk formula along with the allergen(s). Thioredoxin is known to reduce disulphide bonds, and through this activity can lower the allergenicity of certain allergens while maintaining their immunogenicity for OT induction.

The invention also provides a method of treatment or prevention of an allergy by oral tolerance, which comprises orally administering to a patient in need of such treatment an effective amount of a composition comprising milk as a carrier and the exogenous allergen responsible for the allergy. Thus, a composition comprising milk and an exogenous allergen is suitable for use in the treatment or prevention of an allergic syndrome or auto-immune condition resulting from immune reaction to that allergen. The invention therefore also provides the use of a composition comprising milk and an exogenous allergen in the treatment or prevention of an allergic condition or auto-immune disorder associated with that allergen.

Since it is not known what effect immunosuppressive drugs might have on the development of oral tolerance, it is preferred that individuals who are being treated with the milk/allergen mixtures herein described are not concurrently receiving immunosuppressive therapy.

### EXAMPLES

### Example 1

Oral Tolerance Induction in IgE High Responder Dogs

The induction of OT through delivery of antigen in milk was tested in an allergic dog model (de Weck et al. Prakt Tierarzt 1998;79:6-23; and de Weck et al. Allergologie 2, 1999;2:92-97). Fourteen genetically IgE high responder beagles were sensitized to Bet υ 1 antigen (recombinant major birch allergen) by subcutaneous injection on the day of birth and then given three boosters at two-week intervals. All but two of the dogs displayed a positive Bet υ 1-specific IgE titer, confirming their high IgE responder background. The dogs also developed strong Bet υ 1-specific IgG titers, confirming that all dogs were immunized to Bet υ 1.

At 8 weeks the group of fourteen sensitized dogs was split into a control group of 7 dogs and a treatment group of 7 dogs. The treated dogs then received once daily for 4 weeks 100 mg ovalbumin dissolved in 50 ml fresh, pasteurized cow's milk (3.6% fat content); the control group of 7 matched dogs received no treatment during the experimental period. At the age of 12 weeks, following the period of oral tolerance induction, both treated and control dogs were challenged with ovalbumin, β-lactoglobulin and Bet υ 1 by subcutaneous immunization on 4 occasions at two-week intervals.

Serum samples were collected weekly for determination of antigen-specific IgE and IgG levels between the day of oral administration of milk (week 8) and week 20. Thereafter samples were taken weekly between weeks 27 and 34. The sera aliquots were stored at −20° C. before assay by capture ELISAs for ovalbumin-, β-lactoglobulin- and Bet υ 1-specific IgE and IgG levels, respectively.

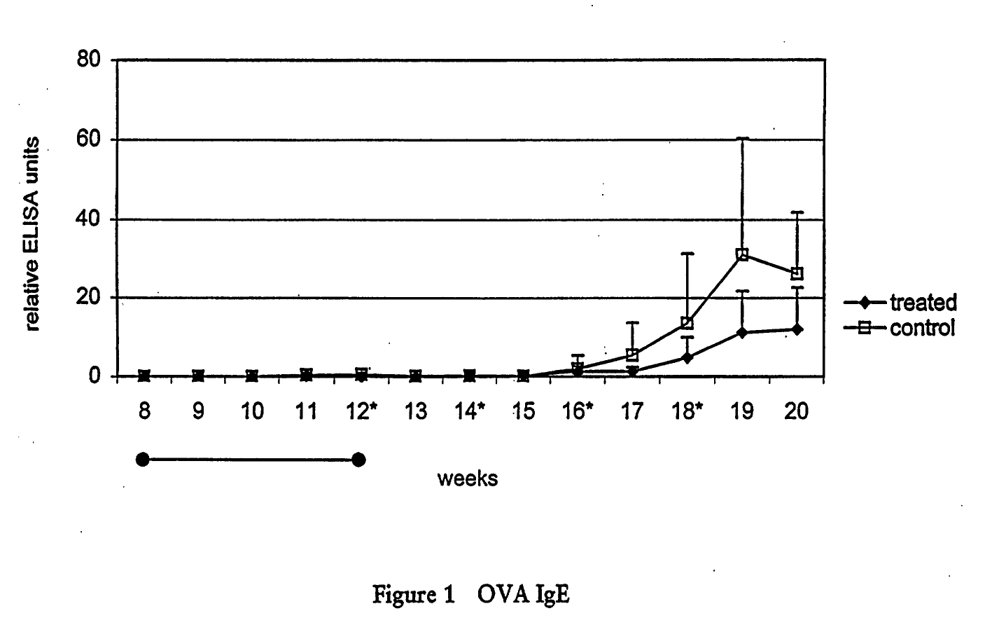

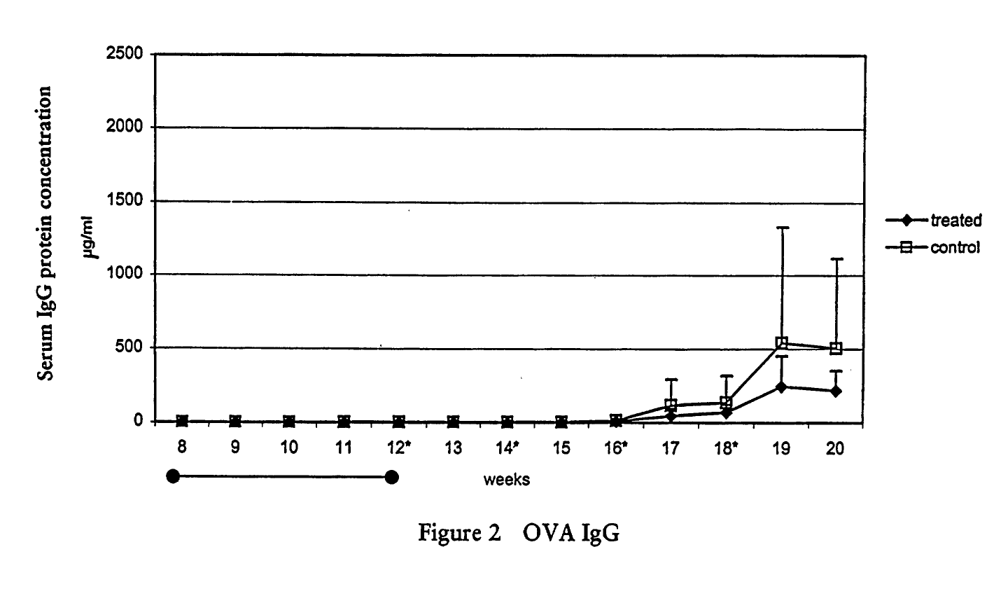

Over the experimental time period, the treated group displayed lower ovalbumin- and β-lactoglobulin-specific serum IgE and IgG ELISA titers than the control group, and the rise in ovalbumin-specific IgE and IgG was delayed compared to control animals, as shown for example in FIGS. 1 and 2. A uniform decline in β-lactoglobulin-specific IgE titer was noted towards the end of the experimental period. The immune response to Bet υ 1 was unaffected by the treatment. No side effects were observed in any of the dogs used in the study.

The experiments were repeated to compare the effects of daily dosages of 10 mg and 100 mg ovalbumin in 50 ml milk over a 21 week period. The effects on OVA-specific serum IgE levels of the daily 10 mg ovalbumin dose were comparable to those associated with the daily 100 mg dose, and in both groups there was a statistically significant reduction in serum OVA-specific IgE compared with the negative control (milk alone).

In addition to measurements of serum IgE and IgG levels, the degree of tolerization achieved by administering 10 mg ovalbumin in the manner described above was further demonstrated by effects at the mucosal level. For example, when compared with the non-treated control group, the treated dogs showed a less severe response to ocular challenge with OVA, and a reduction in the bronchoalveolar eosinophil and neutrophil counts obtained by bronchoalveolar lavage following aerosol challenge with ovalbumin.

In a comparable study, administration of ovalbumin to sensitized dogs in the form of acid-resistant capsules (63 mg ovalbumin per day) was not demonstrated to induce any significant differences in OVA-specific IgG or IgE titers as compared to non-treated control animals, when observed over the course of 82 weeks.

In conclusion, ovalbumin dissolved in milk induces antigen-specific OT in an allergic dog model, both to the allergen carried by the milk, and also to at least one milk allergen. Thus milk appears to be a suitable antigen carrier and presentation substance to induce OT. Without wishing to be limited by theory, one explanation for these promising results is that the coagulation of milk in the digestive system has a protective effect on antigens dissolved in the milk. Another possibility is that the manner of presentation of allergens to the gut-associated lymphoid tissue (GALT) immune system is improved when milk is used as the delivery system.

### Example 2

An infant milk formula useful as a carrier for allergens to induce primary oral tolerance in infants.

100 kcal comprises:

| | | |
|---|---|---|
| Total Fat | 4.6 | g |
| (Linoleic acid) | 0.5 | g |
| (α-linolenic acid) | 0.16 | g |
| Total protein | 2.9 | g |
| (Casein) | 1.18 | g |
| (Albumin/globulin) | 1.77 | g |
| Total carbohydrate | 11.8 | g |
| (glucose) | 2.1 | g |
| (lactose) | 6.1 | g |
| (maltose) | 0.1 | g |
| (maltodextrin) | 2.1 | g |
| (starch) | 1.4 | g |
| Sodium | 41 | mg |
| Potassium | 103 | mg |
| Calcium | 79 | mg |
| Magnesium | 13 | mg |
| Phosphorus | 51 | mg |
| Chloride | 93 | mg |
| Ca:P | 1.5 | |
| Iron | 1.0 | mg |
| Zinc | 1.0 | mg |
| Copper | 62 | μg |
| Iodine | 6.1 | μg |
| Vitamin A | 104 | μg |
| Vitamin B1 | 71 | μg |
| Vitamin B2 | 122 | μg |
| Vitamin B6 | 53 | μg |
| Vitamin B12 | 0.2 | μg |
| Vitamin C | 10.3 | mg |
| Vitamin D3 | 1.2 | μg |
| Vitamin E | 1.0 | mg |
| Vitamin K1 | 4.6 | μg |
| pantothenic acid | 0.7 | mg |
| niacin | 0.5 | mg |
| folic acid | 7 | μg |
| biotin | 1.9 | μg |
| choline | 10.3 | mg |
| taurine | 7.8 | mg |
| cytidine-5′MP-acid | 2.3 | mg |
| uridine-5′MP-Acid | 1.0 | mg |
| adenosine-5′MP-Acid | 0.9 | mg |
| guanosine-5′MP-Acid | 0.4 | mg |
| inosine-5′MP-Acid | 0.2 | mg |

1. Use of milk as a carrier for one or more exogenous allergens for inducing oral tolerance to said allergen(s).

2. A composition comprising a mixture of milk with at least one exogenous allergen other than a composition comprising a mixture of milk with a natural whole foodstuff.

3. A composition according to claim 2 wherein the allergen is substantially pure.

4. A composition according to claim 2 or claim 3 which comprises a cocktail of allergens.

5. A composition according to any of claims 2 to 4 wherein said allergen is not derived from a foodstuff.

6. A composition according to any of claims 2 to 5 which comprises one or more allergens selected from animal dander allergens, house insect allergens, and pollen allergens.

7. A composition according to any of claims 2 to 6 which comprises any combination of allergens selected from: ragweed allergens, house dust mite recombinant allergens Der f1 and Der f2, mature and/or truncated, cat dander allergen Fel d1 (chains 1 and 2), and flea allergen Cte f1.

8. A composition according to any of claims 2 to 7 wherein said milk is cow's milk.

9. A composition according to any of claims 2 to 8 wherein said at least one allergen is not produced through processing by passage through the mammalian body.

10. A composition comprising a mixture of milk with at least one exogenous allergen.

11. A process of preparing a composition according to any of claims 2 to 10 which comprises the step of mixing said milk with said exogenous allergen.

12. A process according to claim 11 which further comprises a preparatory step of extracting, purifying and/or concentrating said allergen from its source.

13. Use of a composition according to any of claims 2 to 10 in the manufacture of a medicament or nutritional formulation for the induction of oral tolerance to said allergen(s).

14. Use according to claim 13 wherein the milk is infant milk formula, and the medicament or nutritional formulation is for induction of primary oral tolerance in infants under the age of 12 months.

15. Use according to claim 13 or claim 14 wherein the medicament or nutritional formulation is for administration to an infant who has a generic predisposition to developing an allergy or auto-immune reaction to said allergen.

16. Use according to claim 13 wherein the milk is cow's milk, and the medicament or nutritional formulation is for induction of secondary oral tolerance in mammals of any age.

17. Use of a composition according to any of claims 2 to 10 in the manufacture of a medicament or nutritional formulation for the treatment or prevention of an allergic condition or auto-immune disease.

18. A method of treatment or prevention of an allergy or auto-immune condition by oral tolerance inductions, which comprises administering to an individual in need of such treatment an effective amount of a composition comprising milk as a carrier and one or more exogenous allergen(s) responsible for the allergy or auto-immune condition.

19. A product (kit) comprising milk, and an allergen or a cocktail of at least two allergens, in separate dosage forms for simultaneous use for the treatment or prevention of an allergy or auto-immune condition caused by said allergen(s).
